(19) United States
(12) Patent Application Publication
(10) Pub. No.: US 2024/0358669 A1
(43) Pub. Date: Oct. 31, 2024

(54) METHODS FOR TREATING PANCREATIC CANCER

(71) Applicant: Olatec Therapeutics, Inc., New York, NY (US)

(72) Inventors: Charles A. Dinarello, Boulder, CO (US); Carlo Marchetti, Denver, CO (US)

(21) Appl. No.: 18/767,860

(22) Filed: Jul. 9, 2024

Related U.S. Application Data
(63) Continuation of application No. PCT/US2023/060234, filed on Jan. 6, 2023.
(60) Provisional application No. 63/266,629, filed on Jan. 10, 2022.

(51) Int. Cl. A61K 31/275; A61K 31/7068; A61P 35/00

(52) U.S. Cl. CPC A61K 31/275; A61K 31/7068; A61P 35/00

(57) ABSTRACT

The present invention provides a method for treating pancreatic cancer. The method comprises administering to a subject in need thereof an effective amount of dapansutrile. Preferred route of administration of dapansutrile is oral administration. The present invention also provides a combination treatment of pancreatic cancer, by administering dapansutrile and gemcitabine to the patient in need thereof.

### CROSS-REFERENCE TO RELATED APPLICATION(S)

This application is a continuation of PCT/US2023/060234, filed Jan. 6, 2023; which claims the benefit of U.S. Provisional Application No. 63/266,629, filed Jan. 10, 2022. The contents of the above-identified applications are incorporated herein by reference in their entirety.

### FIELD OF THE INVENTION

The present invention relates to methods for treating pancreatic cancer by administering an effective amount of dapansutrile to a subject in need thereof.

### BACKGROUND OF THE INVENTION

Pancreatic ductal adenocarcinoma (PDAC) constitutes 90% of pancreatic cancer and is the third leading cause of cancer deaths in the US and seventh worldwide (PMID: 35020204, 30834048). With the reported increase in incidence per year, pancreatic cancer is predicted to become the second leading cause of cancer-related death in the US by 2030 (PMID: 34547082). Lack of effective screening methods and nonspecific symptoms even at advance stage of disease are major limitations in the management of this disease that contribute to the extremely severe prognosis in patient with pancreatic cancer (the 5-year survival reached for the first time 11% in 2022). To date, at diagnosis, only 10-15% of patients are eligible for surgery presenting localized disease while the remaining 85-90% of patients have metastatic (Stage IV) or locally advanced disease which is not suitable for resection (Stage II and III) (PMID: 34547082). For patients with unresectable disease, treatment options are very limited involving radiation and chemotherapeutic regimens including gemcitabine, nab-paclitaxel and FOLFIRINOX. Considering the very modest impact of the standard of care in the overall survival in patients with advance disease and the common serious adverse events associated with these treatments, novel treatment options represent a medical urgency for these patients.

Inflammation has been demonstrated to influence PDAC growth, response to therapy and metastasis with several inflammatory cytokines found increased in patients with PDAC (PMID: 25897428). Of these, the inflammatory cytokine IL-1β has been shown to be upregulated in PDAC, to promote desmoplasia, immune suppression and to correlate to patient's response to chemotherapy (23591198; 31915130; 26500238). IL-1β is initially synthesized as a biologically inactive pro-form which requires processing (ref). Maturation of IL-1β is mostly regulated by conserved cytosolic pattern recognition receptors (PRRs) of the nucleotide-binding and oligomerization domain NOD-like receptor (NLRs) family. In the context of the pancreas, NLRP3, one NLRs member, has shown to mediates inflammation in acute pancreatitis, a recognized high-risk factor for the development of PDAC (PMID: 33228173). Furthermore, NLRP3 signaling has shown to participate in tumor progression in mouse model of PDAC (PMID: 28442553).

There is a need for a method for treating pancreatic cancer. The method should be effective and have no significant side effects.

### BRIEF DESCRIPTION OF THE DRAWINGS

FIG. 1 shows that pancreatic cancer-bearing mice fed with dapansutrile (OLT) diet displayed reduced tumor volume (****=p<0.0001) and reduced tumor weight (**=p<0.01) when compared to mice fed standard (STD) diet (FIG. 1).

FIG. 2 shows that pancreatic cancer-bearing mice treated with dapansutrile (OLT) plus gemcitabine (GEM), significantly further reduced tumor volume and tumor weight comparing with pancreatic cancer-bearing mice treated with OLT alone or GEM alone. ****=p<0.0001, ***=p<0.001, **=p<0.01, *=p<0.05.

### DETAILED DESCRIPTION OF THE INVENTION

The present invention is directed to a method for treating pancreatic cancer such as pancreatic ductal adenocarcinoma using dapansutrile, which is an NLRP3 inhibitor.

NLRP3 (NOD-like receptor family, pyrin domain containing 3), also known as NALP3 or cryopyrin, is one of the sensors of the inflammasome, a macromolecular structure involved in interleukin-1β (IL-1β) and IL-18 processing. NLRP3 activation leads to recruitment of ASC (apoptosis-associated speck-like protein containing carboxyterminal caspase recruitment domain) and caspase-1 leading to inflammasome formation and ultimately cell death.

NLRP3 is highly expressed in pancreatic cancer tissue when compared to normal pancreas. NLRP3 is a cytosolic receptor that, following activation, determines the maturation of the biological inactive inflammatory cytokines IL-1B and IL-18 into their biologically active forms. Once active, NLRP3 forms inflammasome that mediates the processing of pro-IL-1β and pro-IL-18 into their biologically active forms. The inventors have found the formation of the NLRP3 inflammasome in human PDAC samples. Consistently, IL-1β levels are elevated in PDAC samples.

### Compound

The present invention uses a purified compound of dapansutrile (3-methanesulfonyl-propionitrile), or the pharmaceutically acceptable solvate thereof, to treat pancreatic cancer.

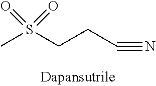

Dapansutrile is a small, synthetic molecule of β-sulfonyl nitrile which has been demonstrated to selectively inhibit the NLRP3 inflammasome and is safe when orally administered to healthy subjects.

“Solvates,” as used herein, are addition complexes in which the compound is combined with an acceptable co-solvent in some fixed proportion. Co-solvents include, but are not limited to, water, acetic acid, ethanol, and other appropriate organic solvents.

### Pharmaceutical Compositions

The active compound dapansutrile, or its pharmaceutically acceptable solvate in a pharmaceutical composition in general is in an amount of about 0.1-5% for an injectable formulation, about 1-90% for a tablet formulation, 1-100% for a capsule formulation, about 0.01-20%, 0.05-20%, 0.1-20%, 0.2-15%, 0.5-10%, or 1-5% (w/w) for a topical formulation, and about 0.1-5% for a patch formulation.

“About” as used in this application, refers to +10% of the recited value.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, gels, and ointments. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cystein, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and ploxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Such pharmaceutically acceptable carriers may be preserved against bacterial contamination using well-known preservatives, these include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of dapansutrile may contain other excipients that have no bioactivity and no reaction with the active compound. Excipients of a tablet may include fillers, binders, lubricants and glidants, disintegrators, wetting agents, and release rate modifiers. Binders promote the adhesion of particles of the formulation and are important for a tablet formulation. Examples of binders include, but not limited to, carboxymethylcellulose, cellulose, ethylcellulose, hydroxypropylmethylcellulose, methylcellulose, karaya gum, starch, starch, and tragacanth gum, poly(acrylic acid), and polyvinylpyrrolidone.

For example, a patch formulation of dapansutrile may comprise some inactive ingredients such as 1,3-butylene glycol, dihydroxyaluminum aminoacetate, disodium edetate, D-sorbitol, gelatin, kaolin, methylparaben, polysorbate 80, povidone, propylene glycol, propylparaben, sodium carboxymethylcellulose, sodium polyacrylate, tartaric acid, titanium dioxide, and purified water. A patch formulation may also contain skin permeability enhancer such as lactate esters (e.g., lauryl lactate) or diethylene glycol monocthylether.

Topical formulations including dapansutrile can be in a form of gel, cream, lotion, liquid, emulsion, ointment, spray, solution, and suspension. The inactive ingredients in the topical formulations for example include, but not limited to, lauryl lactate (emollient/permeation enhancer), diethylene glycol monoethylether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent). In one embodiment, diethylene glycol monoethylether is included in the topical gel formulation.

### Method of Use

By inhibiting assembly of the NLRP3 inflammasome, dapansutrile prevents the production and/or release of proinflammatory cytokines IL-1B, and ultimately reduces pancreatic tumor growth.

The present invention is directed to a method of treating pancreatic cancer, particularly PDAC. The method comprises the step of administering to a subject in need thereof an effective amount of dapansutrile. “An effective amount,” as used herein, is the amount effective to treat a disease by ameliorating the pathological condition, and/or reducing, improving, and/or eliminating the symptoms of the disease. For example, an effective amount is an amount that reduces the growth of pancreatic cancer (reducing tumor size and/or reducing tumor weight).

In one embodiment, a patient suffering from pancreatic cancer is administered with dapansutrile without being administered with immune cells such as cytokine-induced killer cells.

The pharmaceutical composition comprising dapansutrile can be applied by systemic administration or local administration. Systemic administration includes, but is not limited to oral, parenteral (such as intravenous, intramuscular, subcutaneous or rectal), and inhaled administration. In systemic administration, the active compound first reaches plasma and then distributes into target tissues. Oral administration is a preferred route of administration for the present invention. Local administration includes topical administration.

Dosing of the dapansutrile composition can vary based on the extent of the subject's tumor and each patient's individual response. For systemic administration, plasma concentrations of the active compound delivered can vary; but are generally 1×10⁻¹⁰-1×10⁻⁴ moles/liter, and preferably 1×10⁻⁸-1×10⁻⁵ moles/liter.

In one embodiment, the pharmaceutical composition is administrated orally to a subject. The dosage for oral administration is generally at least 0.1 mg drug/kg subject/day and less than 100 mg/kg/day or 200 mg/kg/day. For example, the dosage for oral administration is 1-100, or 5-50, or 10-50 mg/kg/day, for a human subject. For example, the dosage for oral administration is 100-10,000 mg/day, and preferably 500-2000, 500-4000, 500-4000, 1000-5000, 2000-5000, 2000-6000, or 2000-8000 mg/day for a human subject. Dapansutrile can be orally taken once, twice, three times, or four times a day, depending on the patient's age and condition.

In one embodiment, the pharmaceutical composition is administrated intravenously to a subject. The dosage for intravenous bolus injection or intravenous infusion is generally 0.03 to 5 or 0.03 to 1 mg/kg/day.

In one embodiment, the pharmaceutical composition is administrated subcutaneously to the subject. The dosage for subcutaneous administration is generally 0.3-20, 0.3-3, or 0.1-1 mg/kg/day.

In one embodiment, the composition is applied topically. The composition is topically applied at least 1 or 2 times a day, or 3 to 4 times per day, depending on the medical issue and the disease pathology. In general, the topical composition comprises about 0.01-20%, or 0.05-20%, or 0.1-20%, or 0.2-15%, 0.5-10, or 1-5% (w/w) of the active compound. Typically, 0.2-10 mL of the topical composition is applied to the individual per dose.

Those of skill in the art will recognize that a wide variety of delivery mechanisms are also suitable for the present invention.

### Combination Treatment

Considering the limited efficacy of the current treatments for PDAC, it is a pressing issue to find a more effective method to treat human pancreatic cancer.

The present invention is further directed to a method of treating pancreatic cancer by administering dapansutrile and a chemotherapeutic agent. The method comprises the step of administering to a subject in need thereof an effective amount of dapansutrile and an effective amount of a chemotherapeutic agent. Chemotherapeutic agents suitable to be used with dapansutrile for treating pancreatic cancer include, for example, gemcitabine, FOLFIRINOX, and nab-paclitaxel.

FOLFIRINOX is a strong chemotherapy agent that includes leucovorin calcium (folinic acid), fluorouracil, irinotecan hydrochloride, and oxaliplatin.

Nab-paclitaxel is a chemotherapy drug and is also known by its brand name, Abraxane. It combines the chemotherapy drug paclitaxel with a protein called albumin.

Gemcitabine hydrochloride, an intravenously administered chemotherapeutic antimetabolite, is a potent and specific oncolytic used for the treatment of cancer. The antiproliferative mechanism of gemcitabine involves inhibition of DNA replication and repair by inhibiting DNA synthesis and by blocking repair mechanisms through masked chain termination. Gemcitabine blocks proliferation in highly proliferation cells like tumor cells.

The inventors have demonstrated that the combination of dapansutrile with gemcitabine significantly reduced tumor progression when compared to the monotherapies in animals. Gemcitabine is a chemotherapy that prevents DNA synthesis halting the expansion of highly proliferating cells. Dapansutrile blocks NLRP3, increases T cells activity, prevents the PDAC-mediated inflammatory events, and ultimately reduces immunosuppression. Mechanistically, treatment with dapansutrile has no effect in non-immune cells proliferation but has a significant reduction in COX2/PGE2, which signals a potent immunosuppressive pathway in PDAC. The independent targets of gemcitabine and dapansutrile provide the basis for the beneficial effect of the combinational therapy. The combination of these two drugs increases the efficacy of each monotherapy because they combine two different mechanisms to reduce tumor cell growth. The combination treatment of dapansutrile with gemcitabine may reduce the dosage of gemcitabine and provide long-term tolerable treatments for patients with pancreatic cancer.

In one embodiment of the combination treatment, dapansutrile is orally administered daily and the chemotherapeutic agent is administered according to its own standard route and standard dosage, or a lower dosage. For example, gemcitabine is administered intravenously weekly at a dosage of 500-1000 mg/m² surface area or 800-1000 mg/m² surface area.

The present invention is useful in treating a mammal subject, such as humans, horses, dogs and cats. The present invention is particularly useful in treating humans.

The following examples further illustrate the present invention. These examples are intended merely to be illustrative of the present invention and are not to be construed as being limiting.

### EXAMPLES

### Example 1. Dapansutrile Reduced Pancreatic Cancer Growth in Mice

C57BL/6 male mice (8 weeks old) were purchased from Jackson Laboratories (Bar Harbor, ME). Mice were anesthetized and following opening the abdominal cavity, the tip of pancreatic tail was gently grasped, and the pancreas/spleen was externalized in a lateral direction, exposing the entire pancreatic body and spleen. 100,000 KPC cells, a murine PDAC cell line, were mixed 1:1 with Matrigel (VF=20 μl) and injected into the tail of the pancreas. The pancreas was then gently internalized with blunt forceps and the abdominal muscle layer, and the skin was closed separately with absorbable sutures.

Following the initial injections of KPC cells, tumor bearing mice were monitored daily until the sacrifice after 21 days. At sacrifice, tumor volume and weight were assessed in the vehicle (n=16) and OLT1177® (dapansutrile)-treated mice (n=16).

From the date of surgery, mice were treated with oral dapansutrile via feed pellets ad libitum. The composition of the food is the standard mouse chow enriched with 7.5 gr of dapansutrile per kilogram of food. The concentration of dapansutrile in the food is based on the observation that the mean dapansutrile plasma level in mice fed this diet reached the same order of magnitude as the safe level reached in humans in phase I clinical trials (PMID: 30075804).

At sacrifice, tumor volume in different groups was calculated using the modified ellipsoidal formula V=(LW²)/2, where L is the length of the longest tumor dimension parallel to the skin containing the tumor midpoint, W is the length of the tumor dimension perpendicular to L and parallel to the skin, and V is tumor volume in cubic millimeters (mm³).

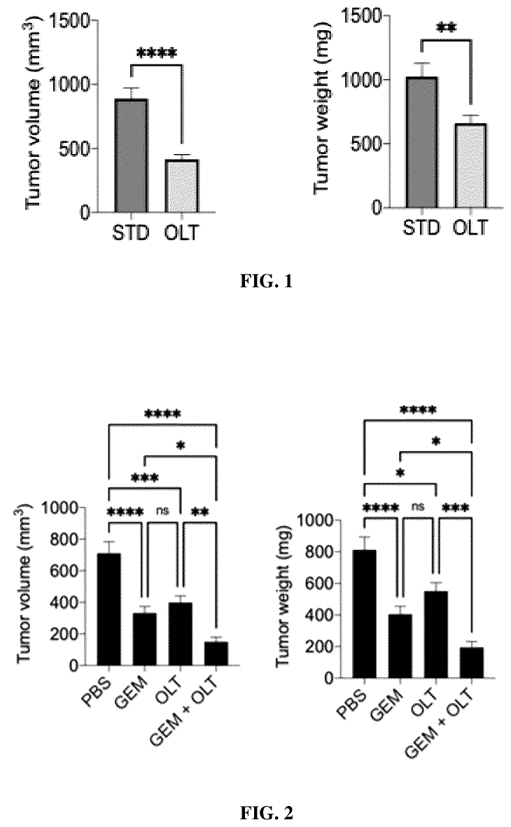

The results show that pancreatic cancer-bearing mice fed with dapansutrile diet displayed reduced tumor volume (****=p<0.0001) and reduced tumor weight (**=p<0.01) when compared to mice fed standard diet (FIG. 1).

### Example 2. Combination Treatment of Dapansutrile and Gemcitabine

Tumor-bearing mice were prepared according to Example 1 and monitored daily until the sacrifice after 21 days.

Four groups of tumor-bearing mice were treated with PBS (n=8), gemcitabine (n=11), dapansutrile (n=8), and dapansutrile plus gemcitabine (n=9). Mice treated with dapansutrile were fed with dapansutrile by the same protocol as described in Example 1. All other three groups of mice had standard diet as described in Example 1.

Gemcitabine (Hospira Inc., Lake Forest, IL) was administrated intraperitoneally (i.p.) at 100 mg/kg every three days starting 10 days after surgery. Control mice received PBS

### Solution

At sacrifice, tumor volume and tumor weight were assessed in the 4 groups of mice. The results are shown in FIG. 2. The results show that gemcitabine alone and dapansutrile alone induced a significant reduction in tumor volume and weight. Mice treated with gemcitabine plus dapansutrile show a significant reduction in tumor volume and tumor weight comparing with vehicle-treated mice and the respective monotherapy (gemcitabine and dapansutrile). The data demonstrate that adding NLRP3 inhibition with dapansutrile to standard of care gemcitabine potentiates the efficacy of gemcitabine alone.

### REFERENCES

1. Y. Guo, et al. Cancer Res 77, 6429-6441 (2017).
2. S. Shalapour, et al. J Clin Invest 125, 3347-3355 (2015).
3. C. A. Dinarello. Blood 117, 3720-3732 (2011).
4. R. N. Apte, et al. Cancer Metastasis Rev 25, 387-408 (2006).
5. C. A. Dinarello. Cancer Metastasis Rev 29, 317-329 (2010).
6. B. Guo, et al. Sci Rep 6, 36107 (2016).
7. C. Marchetti, et al. Proc Natl Acad Sci USA 115, E1530-E1539 (2018).

What is claimed is:

1. A method of treating pancreatic cancer in a subject, comprising the step of:
administering to the subject suffering from pancreatic cancer an effective amount of dapansutrile, or a pharmaceutically acceptable solvate thereof.

2. The method according to claim 1, wherein the pancreatic cancer is pancreatic ductal adenocarcinoma.

3. The method according to claim 1, wherein dapansutrile is administered by systemic administration.

4. The method according to claim 3, wherein the dapansutrile is administered by oral administration.

5. The method according to claim 1, wherein the method reduces the tumor volume or tumor weight.

6. The method according to claim 1, wherein the patient is not administered with cytokine induced killer cells.

7. The method according to claim 1, further comprising administering a chemotherapeutic agent to the subject.

8. The method according to claim 7, wherein the chemotherapeutic agent is gemcitabine.

9. The method according to claim 8, wherein gemcitabine is administered intravenously.
